(19) United States
(12) Patent Application Publication
(10) Pub. No.: US 2007/0299459 A1
(43) Pub. Date: Dec. 27, 2007

(54) Percutaneous Tissue Access Device

(75) Inventors: Bryce Way, San Jose, CA (US); Donald Schomer, Cherry Hills Village, CO (US)

Correspondence Address: CONLEY ROSE, P.C.;David A. Rose, P. O. BOX 3267, HOUSTON, TX, 77253-3267, US

(73) Assignee: X-Sten Corp., Englewood, CO (US)

(21) Appl. No.: 11/426,340

(22) Filed: Jun. 26, 2006

(51) Int. Cl. A61B 17/34

(52) U.S. Cl. 606/185

(57) ABSTRACT

A surgical device for percutaneously accessing a tissue of interest. In an embodiment, the surgical device comprises a handle including a groove. In addition, the surgical device comprises a resilient member disposed within the groove. Further, the surgical device comprises a locking sleeve at least partially disposed within the groove with a first end compressing the resilient member and a second end extending beyond the groove and including a flange defining a locking recess having a non-circular cross-section. Still further, the surgical device comprises a cannula including a locking end, the locking end including a locking flange having a non-circular cross-section adapted to be at least partially received by the locking recess to prevent the rotation of the cannula relative to the locking sleeve. Moreover, the surgical device comprises a means for rotationally coupling the cannula to the handle.

### CROSS-REFERENCE TO RELATED APPLICATIONS

Not Applicable.

### STATEMENT REGARDING FEDERALLY SPONSORED RESEARCH OR DEVELOPMENT

Not Applicable.

### BACKGROUND

1. Field of the Invention

The present invention relates to minimally invasive devices and methods for percutaneously accessing tissue. More particularly, the present invention relates to minimally invasive modular devices and methods for percutaneously accessing tissue. Still more particularly, the present invention relates to minimally invasive modular releasable devices and methods for percutaneously accessing tissue,

2. Background of the Invention

Physicians, veterinarians, or other persons treating a patient (e.g., an individual or animal) often need to access specific tissue(s) of interest (e.g., muscle, fat, bone, organ tissue, etc.) underlying the skin of the patient. For instance, a physician may need to access tissue below the skin of a patient to acquire a tissue sample, to treat a disease in the tissue, or to perform a surgery. In some conventional approaches to access specific tissue(s), an incision is made through the skin and any other intervening tissue (e.g. muscle, fat, bone, etc.) between the skin and the specific tissue of interest and the skin and intervening tissue(s) are typically pushed apart and/or stripped away to expose the specific tissue(s) of interest. For instance, in some conventional approaches to treating some types of spinal stenosis (narrowing of the spinal canal), an incision is made in the back and the muscles and supporting structures are stripped away from the spine, exposing the posterior aspect of the vertebral column. The thickened tissue causing the stenosis (e.g., enlarged ligamentum flavum) is then exposed by removal of a portion of the vertebral arch, often at the laminae, covering the back of the spinal canal (laminectomy). The thickened tissue causing the spinal stenosis (e.g., thickened ligamentum flavum) can then be excised by sharp dissection with a scalpel or punching instruments.

Such conventional procedures to access specific tissue(s) of interest are often performed under general anesthesia. In addition, patients may be admitted to the hospital for several days or more depending on the age and overall condition of the patient. Further, recovery from such invasive procedures may be relatively painful and may require weeks and even months. Still further, often the patients need extended therapy at a rehabilitation facility to regain enough mobility to live independently. Much of the pain and disability following such invasive procedures results from the tearing and cutting of the skin and intervening tissue(s) (e.g., muscles, blood vessels, supporting ligaments, nerves, etc.) that may be necessary to access and expose the specific tissue of interest.

Minimally invasive techniques offer the potential for less post-procedure pain and faster recovery compared to traditional surgeries and approaches to access subcutaneous tissue(s). For example, some percutaneous procedures can be performed with local anesthesia, thereby sparing the patient the risks and recovery time required with general anesthesia. In addition, there is less damage to the intervening tissue(s) when minimally invasive percutaneous techniques are used, thereby reducing pain and preserving much more of the patient's tissue. When minimally invasive procedures are used to correct stenosis, a particular advantage is the preservation of these important stabilizing structures.

A variety of techniques for minimally invasive percutaneous procedures are known. In some conventional percutaneous procedures, a trocar in conjunction with an imaging means (e.g., digital fluoroscopy) may initially be employed to pierce the skin and create a path to the specific tissue(s) of interest. However, if the intervening tissue includes bone (e.g., spinal vertebrae), the trocar may need to be removed in order to permit the insertion of a bone saw into the path created by the trocar to enable cutting of the bone. Following sufficient cutting of the bone, the bone saw may be removed and the trocar reinserted to continue creation of a path to the specific tissue of interest. This process may take several steps requiring withdraw and insertion of various tools and may be time consuming.

Once the path through the skin and intervening tissues is created, the trocar may be removed, leaving behind a cannula if the trocar is used in conjunction with a cannula. The process of separating the trocar from the cannula, to withdraw the trocar while leaving the cannula behind, may require complex manipulation of the trocar tool and/or cannula. For instance, the process of separating the trocar from the cannula may require the use of both of the physician's hands, or require the aid of an attending nurse. Once the trocar is separated from the cannula and removed, the remaining cannula establishes a relatively clear path to the specific tissue of interest that may be used to guide a variety of surgical tools inserted through the cannula to the tissue(s) of interest.

In addition, in some minimally invasive percutaneous procedures, more than one trocar and/or cannula may be employed to access tissue(s) from multiple angles and/or to provide multiple paths to the tissue(s) of interest. For example, some procedures may necessitate the simultaneous use of multiple surgical tools in the subcutaneous tissue of interest. In such a case, each tool may require its own dedicated cannula to access the tissue of interest. In many conventional cannula, a handle generally perpendicular to the cannula is fixed to the end of the cannula and is used by the physician or nurse to manipulate and position the cannula. In some cases, these handles can be relatively bulky and crowd the space available around the cannula opening outside the patient's body. Further, when multiple cannulae are employed, the plurality of handles, one for each cannula, may significantly reduce the space available to the physician or nurse in the general vicinity of the cannula openings outside the patient's body. The crowding may detrimentally affect the physician's ability to perform the procedure, may require additional precautions, and generally presents a nuisance to the physician performing the procedure.

Hence, it remains desirable to provide relatively simple devices and methods for percutaneously accessing specific tissue(s) of interest. It is further desired to provide devices and methods for percutaneously accessing specific tissue(s) of interest that may be sufficiently manipulated and controlled by a single individual or physician without the need for performing complex steps. Still further, it is desired to provide devices and methods for percutaneously accessing specific tissue(s) of interest without significantly reducing the space available to the individual performing the procedure (e.g., physician) proximal the surgical site.

### BRIEF SUMMARY OF SOME OF THE PREFERRED EMBODIMENTS

These and other needs in the art are addressed in one embodiment by a surgical device for percutaneously accessing a tissue of interest. In an embodiment, the surgical device comprises a handle including a longitudinal groove. In addition, the surgical device comprises a resilient member disposed within the groove. Further, the surgical device comprises a locking sleeve at least partially disposed within the groove and slidingly engaging the groove, wherein the locking sleeve has a first end engaging and compressing the resilient member and a second end extending beyond the groove, and wherein the second end includes a flange defining a locking recess having a non-circular cross-section. Still further, the surgical device comprises a cannula including a locking end and an axial through bore, wherein the locking end includes a locking flange having a non-circular cross-section. The non-circular cross-section of the locking flange and the non-circular cross-section of the locking recess are adapted to prevent the rotation of the cannula relative to the locking sleeve when the locking flange is at least partially received by the locking recess. Moreover; the surgical device comprises a means for rotationally coupling the cannula to the handle.

Theses and other needs in the art are addressed in another embodiment by a surgical device for percutaneously accessing a tissue of interest. In an embodiment, the surgical device comprises a handle including an axial annular groove defining an axial post, wherein the axial post has a threaded portion. In addition, the surgical device comprises a resilient member disposed within the annular groove. Further, the surgical device comprises an annular locking sleeve partially disposed within the annular groove, wherein the annular locking sleeve has a first end engaging and compressing the resilient member and a second end extending beyond the annular groove, wherein the second end includes a flange having a locking recess with a non-circular cross-section. Still further, the surgical device comprises a cannula including a locking end and an axial through bore, wherein the locking end includes a locking flange having a non-circular cross-section, and wherein the axial through bore has an threaded portion adapted to mate with the threaded portion of the axial post. The non-circular cross-section of the locking flange and the non-circular cross-section of the locking recess are adapted to prevent the rotation of the cannula relative to the locking sleeve when the locking flange is at least partially received by the locking recess. Moreover, the surgical device has a locked position with the cannula threadibly coupled to the axial post and the locking flange at least partially received within the locking recess,

Theses and other needs in the art are addressed in another embodiment by a surgical device for percutaneously accessing a tissue of interest. In an embodiment, the surgical device comprises a handle including an axial annular groove defining an axial post. In addition, the surgical device comprises a resilient member disposed within the annular groove. Further, the surgical device comprises an annular locking sleeve at least partially disposed within the annular groove and slidingly engaging the axial post, wherein the annular locking sleeve has a first end engaging and compressing the resilient member and a second end extending beyond the annular groove Still further, the surgical device comprises a cannula including a locking end and a central axial bore. Moreover, the surgical device comprises a means of rotationally coupling the locking end of the cannula to the post. Additionally, the surgical device comprises a means of restricting the rotation of the locking end relative to the annular sleeve.

Thus, embodiments described herein comprise a combination of features and advantages intended to address various shortcomings associated with certain prior devices. The various characteristics described above, as well as other features, will be readily apparent to those skilled in the art upon reading the following detailed description of the preferred embodiments, and by referring to the accompanying drawings. It should be appreciated by those skilled in the art that the conception and the specific embodiments disclosed may be readily utilized as a basis for modifying or designing other structures for carrying out the same purposes of the embodiments described herein. It should also be realized by those skilled in the art that such equivalent constructions do not depart from the spirit and scope of the invention as set forth in the appended claims.

### BRIEF DESCRIPTION OF THE DRAWINGS

For a more complete understanding of the invention, reference is made to the accompanying drawings, wherein:

FIG. 1 is side view of a percutaneous tissue access device constructed in accordance with a first embodiment of the present invention in a locked position;

FIG. 2 is a cross-sectional view of the percutaneous tissue access device of FIG. 1;

FIG. 3 is a cross-sectional view of the handle and elongate member of FIG. 1;

FIG. 4 is an end view of the handle and elongate member of FIG. 1;

FIG. 5 is a side view of the locking sleeve of FIG. 1;

FIG. 6 is a top view of the locking sleeve of FIG. 1;

FIG. 7 is a cross-sectional view of the locking sleeve of FIG. 1;

FIG. 8 is a side view of the cannula of FIG. 1;

FIG. 9 is an end view of the cannula of FIG. 1;

FIG. 10 is a cross-sectional view of the cannula of FIG. 1;

FIGS. 11-13 are selected cross-sectional views of the percutaneous tissue access device of FIG. 1 in various unlocked positions;

FIG. 14 is cross-section of the spine viewed from the space between two vertebrae, showing the upper surface of one vertebra and the spinal canal with the dural sac and a normal (un-stenosed) ligamentum flavum therein;

FIG. 15 is an illustration of the same section as FIG. 14, showing the spinal canal with the dural sac and a thickened ligamentum flavum therein;

FIG. 16 is a partial cross-section of the lumbar portion of the vertebral column taken along lines 6-6 in FIG. 14;

FIG. 17 is the cross-section of FIG. 16, showing the orientation of an imaging tool relative to the vertebral column; and

FIG. 18 is the cross-section of FIG. 16, showing the orientation of the percutaneous tissue access device of FIG. 1 relative to the vertebral column.

### DETAILED DESCRIPTION OF THE PREFERRED EMBODIMENTS

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Certain terms are used throughout the following description and claims to refer to particular features or components. As one skilled in the art will appreciate, different persons may refer to the same feature or component by different names. This document does not intend to distinguish between components or features that differ in name but not function. The drawing figures are not necessarily to scale. Certain features and components herein may be shown exaggerated in scale or in somewhat schematic form and some details of conventional elements may not be shown in interest of clarity and conciseness.

In the following discussion and in the claims, the terms “including” and “comprising” are used in an open-ended fashion, and thus should be interpreted to mean including, but not limited to . . . .” Also, the term “couple” or “couples” is intended to mean either an indirect or direct connection. Thus, if a first device couples to a second device, that connection may be through a direct connection, or through an indirect connection via other devices and connections,

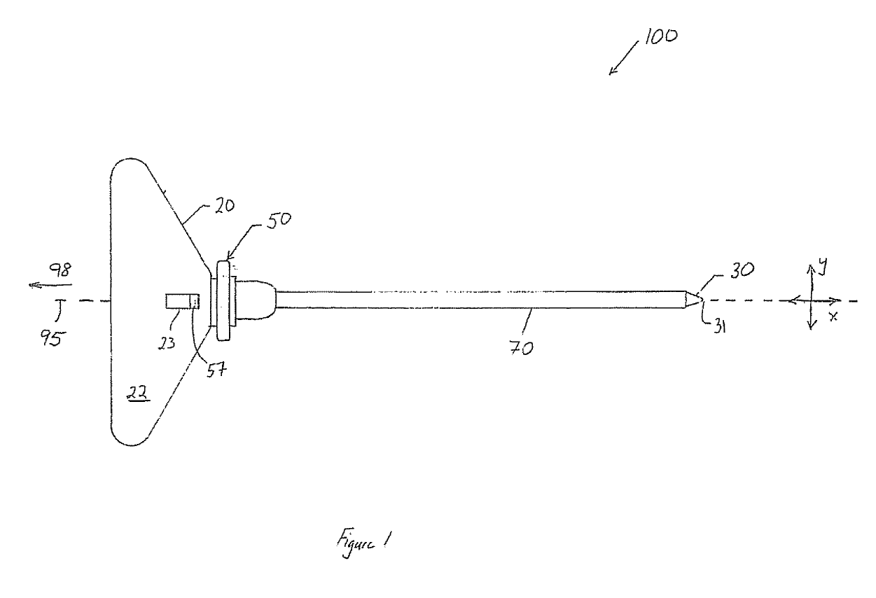

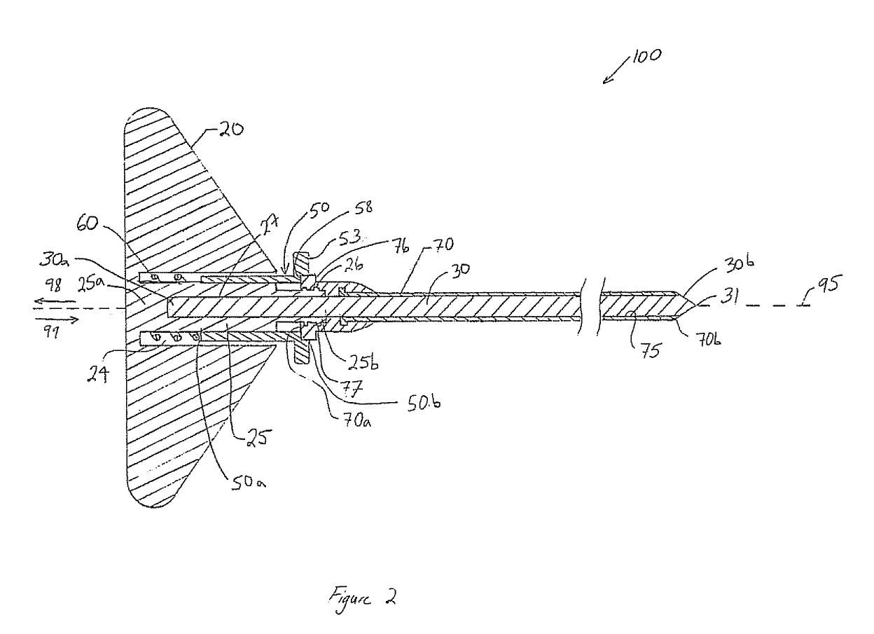

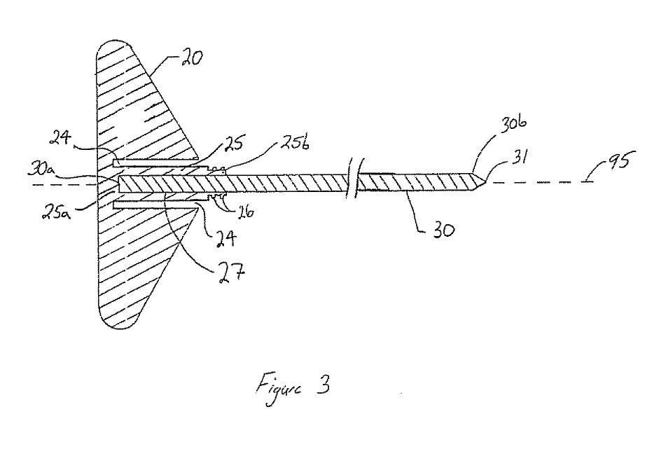

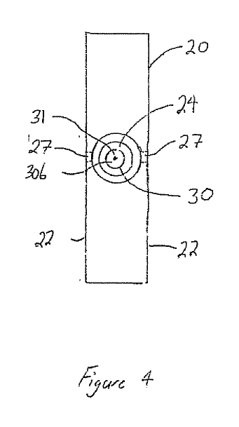

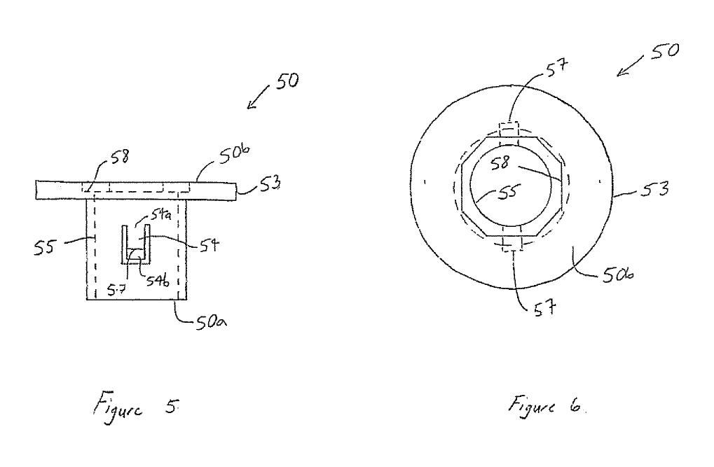

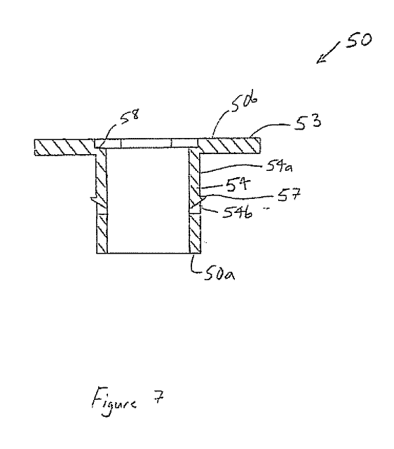

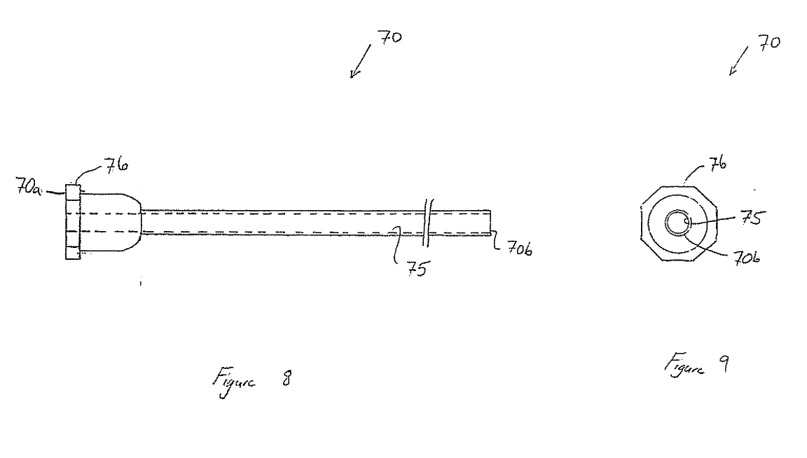

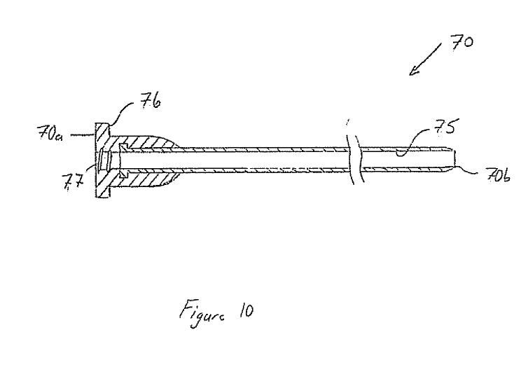

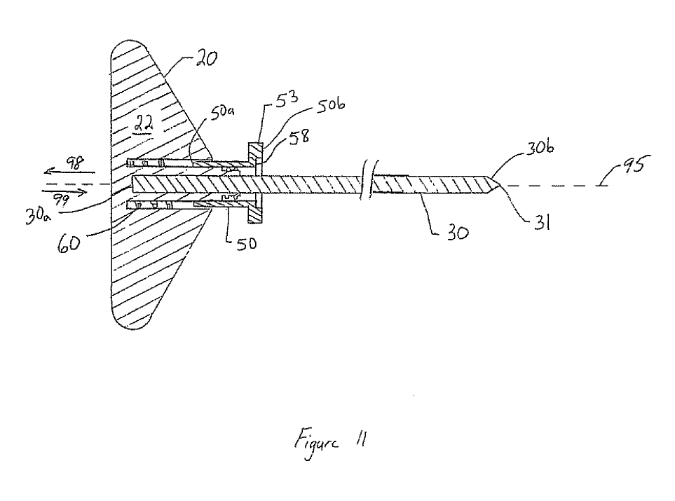

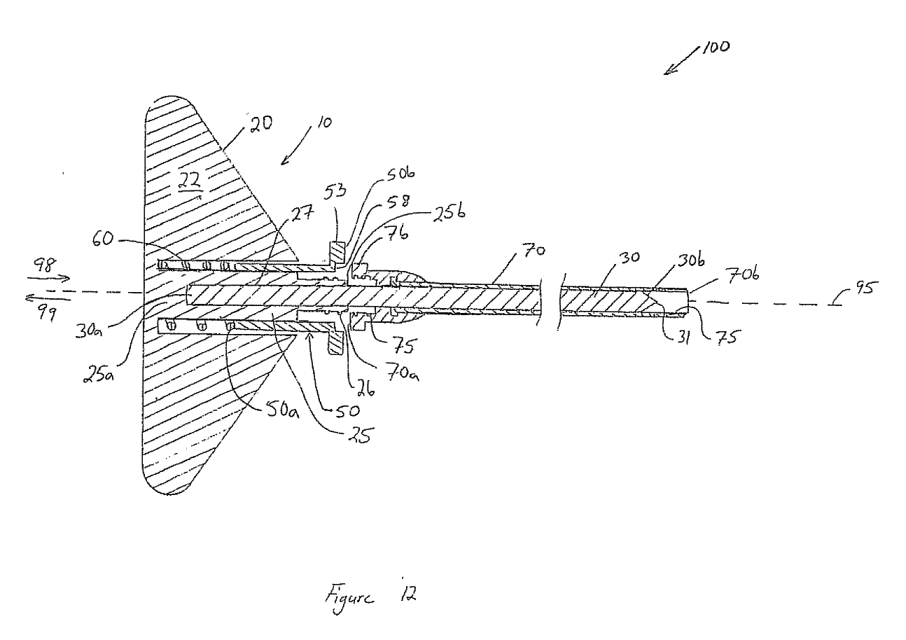

FIGS. 1 and 2 illustrate a first embodiment of a percutaneous tissue access device 100 in accordance with the present invention. Device 100 comprises a handle 20, an elongate member 30, an annular locking sleeve 50, and a cannula 70. Elongate member 30 is rotationally and axially fixed to handle 20 and extends axially from handle 20 through cannula 70. Locking sleeve 50 slidingly engages handle 20 and is permitted to move axially relative to handle 20 and cannula 70. Further; handle 20 is releasably and rotatably coupled to cannula 70, such that handle 20 and cannula 70 may be decoupled and completely separated by rotation of cannula 70 relative to handle 20. In general, device 100 is employed by a user (e.g., physician, nurse, veterinarian, or other individual) to percutaneously access a targeted tissue of interest of a patient (e.g., individual, animal, etc.) as described in detail below. Such targeted tissue(s) may include without limitation muscle, tendon, ligaments, soft tissue, organs, bone, blood vessels, or combinations thereof.

When assembled, device 100, cannula 70, and elongate member 30 have a common longitudinal axis 95. For purposes of the discussion to follow, positions and movement on or substantially parallel to axis 95 are generally described as “axial” or “axially”, while positions and movement substantially perpendicular to axis 95 are generally described as “radial” or “radially.”

FIGS. 1 and 2 illustrate device 100 in the “locked” position with cannula 70 rotatably coupled to handle 20 and restricted from rotating relative to handle 20 by locking sleeve 50. When device 100 is in the “locked” position, device 100 is generally ready to be employed to percutaneously access a tissue of interest in the patient. Thus, as used herein, the term “locked” refers to configurations of device 100 in which cannula 70 is rotatably coupled to handle 20 and prevented from rotating relative to handle 20 by locking sleeve 50. When device 100 is in an “unlocked” position, cannula 70 is not restricted from rotating relative to handle 20 by locking sleeve 50, and thus cannula 70 is allowed to be rotated relative to handle 20, thereby decoupling cannula 70 from handle 20. Several illustrations of device 100 in “unlocked” positions are shown in FIGS. 11-13, which will be discussed in more detail below.

Referring to FIGS. 2-4, device 100 is generally T-shaped, with elongate member 30 and cannula 70 coaxially positioned along axis 95 and handle 20 extending substantially perpendicularly to axis 95. In general, handle 20 is grasped by the user of device 100 and used to orient, position, and guide elongate member 30 and cannula 70 as they are advanced through the skin and into the subcutaneous tissue(s) of the patient. Handle 20 is shown as triangular is shape and having generally parallel lateral sides 22. However, in general, handle 20 may comprise any suitable shape or geometry that provides a functional structure by which the user may grasp, manipulate, and control device 100 during use. Further, handle 20 is preferably ergonomically shaped such that device 100 may be easily handled and employed by a single hand of the user,

The distal face of handle 20 includes an axial annular groove 24 that defines an axial cylindrical post 25. Annular groove 24 may be cast or molded into handle 20, machined from handle 20, or formed by other suitable means. Although groove 24 is described as annular, as will be explained below, groove 24 may comprise any suitable geometry including without limitation a circular groove or slot, a rectangular groove or slot, a longitudinal groove or slot, or combinations thereof.

Post 25 has a first end 25a that is integral with handle 20 and a second end 25b that extends axially beyond groove 24 and the remainder of handle 20. Post 25 further includes threads 26 around its outer radial surface proximal second end 25b. Specifically, threads 26 are employed to releasably couple post 25 to cannula 70. Threads 26 are preferably configured and spaced for relatively easy threading and unthreading with mating threads provided in cannula 70. Although post 25 is described as integral with handle 20, alternatively, post 25 may be manufactured as a separate component that is coupled to handle 20 at first end 25a.

Referring briefly to FIGS. 1 and 4, handle 20 further includes two slots or apertures 23 that extend through each lateral side 22 to annular groove 24. Apertures 23 are used to slidingly couple handle 20 to locking sleeve 50, thereby permitting axial movement of annular locking sleeve 50 relative to handle 20 within groove 24, while at the same time restricting a complete decoupling of locking sleeve 50 and handle 20. As will be described in more detail below, locking sleeve 50 includes projections configured to extend into apertures 23 in handle 20 and adapted to move axially within apertures 23.

Referring again to FIGS. 2-4, elongate member 30 is generally an elongate cylindrical tool extending axially from handle 20. Elongate member 30 has a fixed end 30a coupled to handle 20 and a free end 30b that is distal to handle 20. Specifically, fixed end 30a and an axial portion of elongate member 30 proximal fixed end 30a are disposed within a cylindrical axial bore 27 provided in post 25 of handle 20. Elongate member 30 is preferably partially disposed within bore 27 and fixed to handle 20 within bore 27 such that elongate member 30 does not move rotationally or axially relative to handle 20. Elongate member 30 may be fixed to handle 20 within bore 27 by any suitable means including without limitation mating threads, an adhesive, a locking mechanism, interference fit, shrink fit, or combinations thereof. Although handle 20 and elongate member 30 are described herein as separate components that are fixed together, in different embodiments, handle 20 and elongate member 30 may be formed as a single integral unit.

In the embodiments illustrated herein, elongate member 30 is a trocar having a relatively sharp point or tip 31 adapted to pierce the skin of the patient and traverse subcutaneous tissue(s) of the patient. Such a trocar used in device 100 may be any trocar known in the art. However, in different embodiments, elongate member 30 may be any suitable tool or device including without limitation a cannula, a bone saw, a bone drill bit, etc.

Referring to FIGS. 2 and 5-7, annular locking sleeve 50 comprises a first end 50a, a second end 50b, and a central through bore 55 extending the entire axial length of locking sleeve 50. Locking sleeve 50 preferably further includes two tabs 54 each having a cantilevered end 54a integral with locking sleeve 50 and a free-end 54a including a radial extension or projection 57 as best seen in FIGS. 5 and 7. Each projection 57 extends radially from the outer radial surface of locking sleeve 50. Each projection 57 is sized and configured to mate and slidingly engage with an aperture 23 provided in each lateral side 22 of handle 20.

Second end 50b of locking sleeve 50 includes a radially extending flange 53. In the embodiment illustrated in FIG. 6, flange 53 has a circular cross-section, however, in general, flange 53 may have any suitable geometry including without limitation circular, rectangular, etc. Locking sleeve 50 is moved axially by the user by physically contacting and pushing axially on flange 53. Thus, flange 53 preferably provides second end 50b with sufficient surface area to permit the user of device 100 to push axially on second end 50b generally in the direction of arrows 98 with one or two fingers of the same hand holding handle 20.

Referring specifically to FIGS. 5-7, flange 53 also includes a locking recess 58 provided in second end 50b. In the embodiment shown, locking recess 58 has an octagonal cross-section. As will be explained in more detail below, locking recess 58 has a cross-sectional shape adapted to engage a mating end of cannula 70, thereby restricting rotation of cannula 70 relative to handle 20 when device 100 is in the “locked” position. It should be understood that although locking recess 58 is shown as having an octagonal cross-section, in general, locking recess 58 may have any suitable non-circular cross-section for engaging an end of cannula 70 to restrict rotation of cannula 70 relative to handle 20.

Referring to FIG. 2, locking sleeve 50 is slidingly disposed within annular groove 24 provided in handle 20. Specifically, locking sleeve 50 is disposed in annular groove 24 with post 25 disposed through and slidingly engaging bore 55. Although first end 50a of locking sleeve 50 is disposed within groove 24, second end 55b extends axially beyond groove 24 with flange 53 positioned outside groove 24 distal handle 20. In this configuration, second end 50b and flange 53 are not disposed within groove 24 and may be positively engaged by the user of device 100.

Locking sleeve 50 is sufficiently aligned and disposed within groove 24 such that each projection 57 extending radially from locking sleeve 50 engages a mating aperture 23 provided in handle 20. With projections 57 disposed in apertures 23, locking sleeve 50 is permitted to move axially generally in the direction of arrows 98 and 99 relative to handle 20 and post 25, however, apertures 23 engage projections 57, and restrict locking sleeve 50, from disengaging from handle 20 and post 25. In addition, with projections 57 disposed in apertures 23, locking sleeve 50 is restricted from rotating about axis 95 relative to post 25 and handle 20.

Still referring to FIG. 2, a resilient member 60 is disposed in groove 24 between locking sleeve 50 and handle 20 around post 25. When locking sleeve 50 is in its normal use positions, resilient member 60 is slightly compressed between locking sleeve 50 and handle 20 such that resilient member 60 contacts first end 50a of locking sleeve 50 and tends to push locking sleeve 50 and handle 20. Put another way, resilient member 60 exerts axial forces on locking sleeve 50, urging it out of groove 24 and generally in the direction of arrows 99. However, as previously discussed, engagement of mating projections 57 with apertures 23 prevents the separation of locking sleeve 50 and handle 20. With the exertion of axial forces generally in the direction of arrow 98 on locking sleeve 50 (e.g., exertion of axial forces by the user of device 100 on the surface of flange 53), locking sleeve 50 may be pushed axially further into groove 24 until flange 53 contacts handle 20. However, as locking sleeve 50 is pushed axially further into groove 24, resilient member 60 exerts a restoring or biasing force generally in the direction of arrow 99 that opposes the axial movement. In the embodiments described herein, resilient member 60 is a coil spring, however it will be understood that, in general, resilient member 60 may be any mechanism or device capable of exerting restoring or biasing forces on locking sleeve 50 including without limitation a compressible elastomer, a cantilevered spring, an air spring, or combinations thereof

Although locking sleeve 50 and groove 24 have been described as “annular”, it should be understood that locking sleeve 50 and groove 24 may comprise any suitable shape or geometry permitting locking sleeve 50 to slidingly engage groove 24 and handle 20. For instance, in some embodiments, a rectangular locking sleeve slidingly engages a mating rectangular groove provided in handle 20. In other embodiments, handle 20 may comprise one or more longitudinal grooves or slots adapted to accommodate one or more mating extensions of a locking sleeve. For example, handle 20 may include a longitudinal slot within which a mating extension of a locking sleeve are partially disposed, thereby enabling sliding engagement between the locking sleeve and handle 20. In such embodiments, a resilient member may be disposed within the one or more longitudinal grooves or slots in handle 20 between handle 20 and the locking sleeve.

Referring to FIGS. 2 and 8-10, cannula 70 comprises a locking end 70a, a free end 70b, and a central axial through bore 75. Free end 70b of cannula 70 is generally distal handle 20. The edges of flee end 70b are preferably sharpened to enhance the ability and efficiency by which flee end 70b and cannula 70 cut through subcutaneous tissue(s).

Locking end 70a includes a locking flange 76 having an octagonal cross-section adapted to engage with locking recess 58 of locking sleeve 50. When locking flange 76 is partially received in and engages locking recess 58, locking flange 76 is restricted from rotating about axis 95 relative to locking recess 58. Although locking flange 76 and locking recess 58 are shown and described as having mating octagonal cross-sectional geometries, in general, locking flange 76 and locking recess 58 may have each have any suitable non-circular cross-sectional geometry that enables locking recess 58 to engage and restrict rotation of locking flange 76 when locking flange 76 is at least partially received within locking recess 58. For instance, locking flange 76 and locking recess 58 may have mating rectangular cross-sections. As another example, locking flange 76 may have a rectangular cross-section and locking recess 58 may have an oval cross-section. It should be understood that a circular cross-section for either locking flange 76 or locking recess 58 may not sufficiently restrict the rotation of locking flange 76 relative to locking recess 58 when locking flange 76 is partially received within locking recess 58. For example, if locking recess 58 has a circular cross-section and locking flange 76 has a hexagonal cross-section, then locking flange may still be able to rotate relative to locking recess 58 even when locking flange 76 is partially disposed within locking recess 58. However, other rotation-preventing means are contemplated, including the use of tabs, pins, or bosses and corresponding recesses, or other combinations that prevent relative rotation once axial movement of locking flange 76 allows their respective components to engage.

As best seen in FIG. 10, the inside surface of bore 75 of cannula 70 includes a set of threads 77 proximal locking end 70a. Threads 77 are configured to mate with threads 26 of post 25, thereby enabling the rotational coupling of cannula 70 with handle 20. By properly engaging mating threads 26, 77 and rotating cannula 70 relative to post 25, cannula 70 may be coupled to handle 20 or decoupled from handle 20, depending on the direction of rotation.

Although cannula 70 is illustrated in FIG. 10 as comprising two components fixed together to form a unitary structure, cannula 70 may alternatively be formed as a single integral part including the cannula body 90, bore 75, and locking flange 76.

Referring to FIG. 2, elongate member 30 is slidingly disposed in bore 75 of cannula 70. In addition, cannula 70 is rotatably coupled to post 25 via mating threads 26, 77, which restrict axial movement of cannula 70 relative to handle 20. Further, locking flange 76 is at least partially received within locking recess 58, which restricts rotation of locking flange 76 relative to locking recess. Thus, axial movement of cannula 70 relative to handle 20 is restricted by engagement of mating threads 26, 77, and rotational movement of cannula 70 relative to handle 20, which may otherwise result in decoupling of mating threads 26, 77, is restricted by engagement of locking flange 76 and locking recess 58.

Although cannula 70 is described as being coupled to post 25 of handle 20 by mating threads 26, 77, in general, cannula 70 may be coupled to post 25 by any suitable rotational coupling including without limitation mating threads, a bayonet mechanism, or bayonet-type coupling. As used herein, “bayonet mechanism” or “bayonet coupling” refers to any connection involving a male end having at least one projection in which the male end engages with a female end which has mating slots to the at least one projection of the male end. A bayonet mechanism usually involves rotating the male end less than about 180° in order to lock or secure the connection. It is designed for rapid coupling and decoupling, involving the turning of one part through a small arc, as an alternative to a mating threads connection which may requires one or more full turns to achieve a sufficient coupling.

### Assembly of Percutaneous Tissue Access Device

Prior to use, the various components of percutaneous tissue access device 100 (e.g., handle 20, elongate member 30, locking sleeve 50, cannula 70, etc.) are assembled and configured into the “locked” position illustrated in FIG. 2.

Device 100 is preferably assembled for use as follows: referring to FIG. 11, resilient member 60 is completely disposed within annular groove 24 around post 25. Once resilient member 60 is sufficiently disposed in annular groove 24, locking sleeve 50 is inserted into groove 24, first end 50a first, with projections 57 generally aligned with apertures 23 in handle 20. Locking sleeve 50 is pushed into groove 24 until projections 57 pass through and engage apertures 23 in handle 20. Locking sleeve 50, groove, 24, and resilient member 60 are sized such that resilient member 60 is in compression when projections 57 engage apertures 23. Thus, as long as projections 57 are disposed in apertures 23, resilient member 60 will be in compression, thereby exerting axial forces on locking sleeve 50 generally in direction of arrow 99. As previously discussed, although resilient member 60 exerts axial forces in the direction of arrow 99, positive engagement of projections 57 and apertures 23 limit the axial movement of locking sleeve 50 relative to post 25.

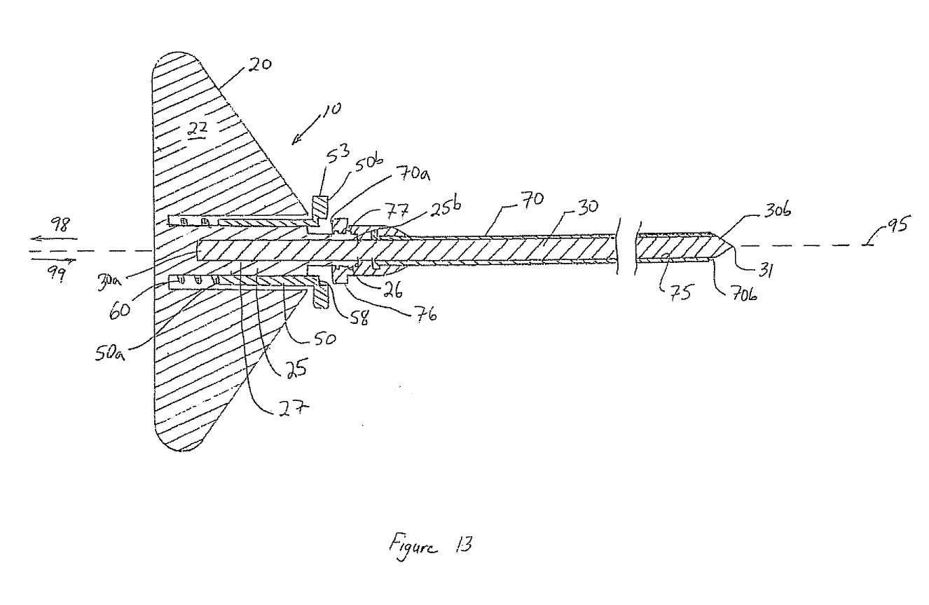

Referring now to FIG. 12, elongate member 30 is inserted and axially advanced into bore 75 of cannula 70. Once elongate member 30 is sufficiently disposed within bore 75, locking end 70a of cannula 70 is positioned adjacent flee end 25b of post 25. At this point, locking flange 76 is not received within locking recess 58, and thus cannula 70 is free to rotate about axis 95 relative to post 25. Cannula 70 is urged toward post 25 and rotated to initialize engagement of mating threads 26, 77. However, continued threading of mating threads 26, 77 is restricted once locking flange 76 is partially received within and engages locking recess 58, thereby restricting further threading of mating threads 26, 77. However, by urging flange 53 of locking sleeve 50 in the direction of arrow 98 with sufficient force to over come resilient member 60, locking sleeve 50 and locking recess 58 may be moved axially away from locking flange 76 of cannula 70 as best seen in FIG. 13. Once locking recess 58 is positioned axially apart from locking flange 76, and therefore locking recess 58 does not engage locking flange 76, cannula 70 is free to rotate relative to post 25, thereby permitting continued threading of mating threads 26, 77 coupling cannula 70 and post 25. As previously discussed, in other embodiments, cannula 70 may be rotationally coupled to post 25 by a bayonet-type coupling.

Referring now to FIG. 2, once cannula 70 is sufficiently secured to post 25 via mating threads 26, 77, exertion of forces in the direction of arrow 99 on flange 53 may cease, allowing resilient member 60 to exert a restoring force on locking sleeve 50 in the direction of arrow 98. Such restoring force pushes locking sleeve 50 axially in the direction of arrow 98 generally until locking flange 76 is at least partially received within locking recess 58. Minor rotation of locking flange 76 may be necessary to properly align locking flange 76 with recess 58 in order to permit locking flange 76 to be received within locking recess 58. Engagement of locking recess 58 and locking flange 76 restricts rotation of cannula 70 relative to post 25, thereby restricting further threading or unthreading of mating threads 26, 77.

Still referring to FIG. 2, device 100 is fully assembled and cannula 70 is rotationally and axially locked in place by the combined effects of mating threads 26, 77 and engagement of locking recess 58 and locking flange 76. Mating threads 26, 77 restrict axial movement of cannula 70 relative to handle 20 as long as cannula 70 is restricted from rotating about axis 95 relative to handle 20. Engagement of locking recess 58 and locking flange 76 restricts such rotation. In this manner, cannula 70 is releasably coupled and locked to handle 20,

### Use of Percutaneous Tissue Access Device

Percutaneous tissue access device 100 is ready for use on a patient when device 100 is properly assembled and “locked” as illustrated in FIG. 2. Device 100 is preferably employed by a user to percutaneously access tissue(s) of a patient as follows: the user of device 100 grasps handle 20 and positions device 100 and tip 31 of elongate member 30 (e.g, trocar) to have a trajectory across the subcutaneous tissue(s) of interest. Then, by pushing on handle 20, the user may puncture the skin of the patient with tip 31. With continued exertion of axial forces on handle 20 and with the aid of imaging means (e.g., digital fluoroscopy), elongate member 30 and cannula 70 may be advanced through the subcutaneous tissue of the patient towards the tissue(s) of interest. Following insertion of tip 31 and/or during advancement of elongate member 30 and cannula 70 into the patient, the orientation and alignment of device 100 may be slightly adjusted as necessary by manipulating handle 20. In general, tip 31 of elongate member 30 and second end 70b of cannula 70 are advanced into the patient until second end 70b of cannula 70 is sufficiently positioned adjacent or into the tissue(s) of interest. Although second end 70b of cannula 70 is positioned adjacent or in the subcutaneous tissue(s) of interest, first end 70a of cannula 70, locking sleeve 50, and handle 20 are positioned outside the patient's body, with cannula 70 and elongate member 30 passing into the patient. Although cannula 70 is properly positioned, elongate member 30 is disposed within bore 75 and thus prevents the insertion of surgical tools through bore 75. Thus, elongate member 30 is preferably withdrawn from cannula 70 once cannula 70 is properly positioned.

Referring to FIG. 13, to disengage cannula 70 from post 25 so that handle 20 and elongate member 30 may be withdrawn, leaving cannula 70 properly in place, locking sleeve 50 must be move axially relative to cannula 70 in the direction of arrow 98, thereby disengaging locking recess 58 and locking flange 76. To perform this step, the user holding handle 20 may simply place two fingers across the surface of locking flange 53 and squeeze, thereby pushing locking sleeve 50 further into groove 24 and disengaging locking recess 58 with locking flange 76. Upon disengagement of locking recess 58 and locking flange 76, cannula 70 may be rotated relative to post 25 to unthread mating threads 26, 77.

Referring to FIG. 12, mating threads 26, 77 have been sufficiently unthreaded such that cannula 70 and post 25 are no longer secured together. By axially pulling handle 20 in the direction of arrow 98, elongate member 30 may then be withdrawn from bore 75 of cannula 70. Handle 20 is pulled until elongate member 30 is completely withdrawn, leaving cannula 70 properly positioned with first end 70a outside the patient, second end 70b positioned adjacent or in the subcutaneous tissue(s) of interest, and bore 75 completely open. Various tools and devices may then be inserted through bore 75 to perform various procedures in the tissue(s) of interest. In this manner, device 100 and cannula 70 provide percutaneous access to tissue(s) of interest in a patients

Each component of device 100 (e.g., handle 20, elongate member 30, cannula 70, locking sleeve 50, etch) may comprise any suitable material including without limitation metals (titanium, aluminum, stainless steel, etc.), non metals (polymers, elastomers, composites, etc.) or combinations thereof. Those components inserted into the patient (e.g., elongate member 30, cannula 70, etc.) preferably comprise a bio-compatible material (e.g., titanium, stainless steel, plastic, etc.).

In this manner, device 100 may be used to provide percutaneous access to tissue(s) of interest in a patient. It should be appreciated that the operation of device 100 is relatively simple and may be performed with one hand of the user, potentially reducing the complexity and number of steps conventionally required to access subcutaneous tissue(s). Further, once elongate member 30 is completely withdrawn from cannula 70, only cannula 70 remains positioned through the patient's skin. Handle 20 is no longer coupled to cannula 70 and no longer takes up space or provides a nuisance near first end 70a of cannula 70. In cases when multiple cannula 70 are employed to percutaneously access tissue(s) of interest, the elimination of handles 20 among the cannula and adjacent the openings in the cannula (e.g., bore 75 at first end 70a), offers the potential to significantly increase the available space around the plurality of cannula within which the user may work.

### Use of Percutaneous Tissue Access Device to Treat Spinal Stenosis

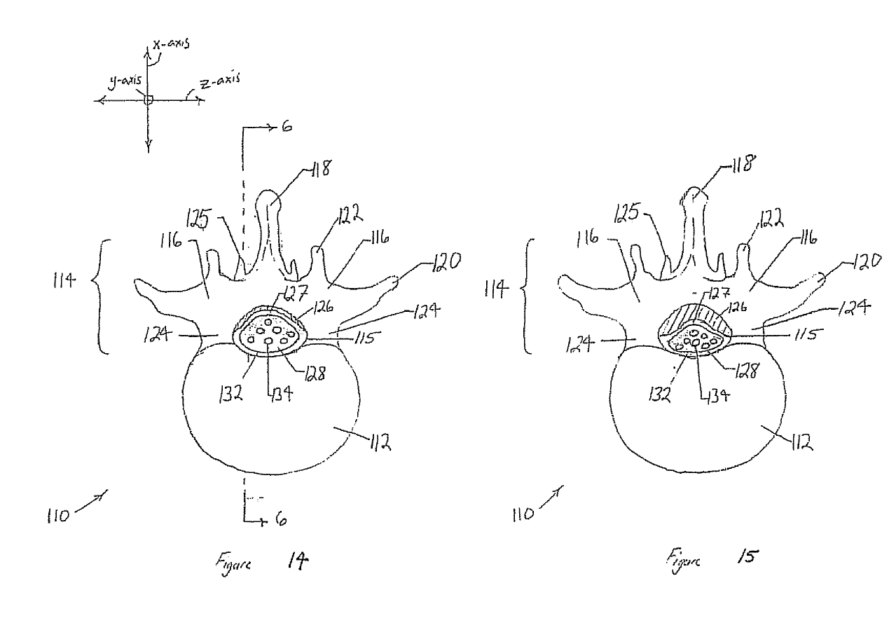

For purposes of the following discussion, the x-, y-, and z-axes are shown in FIGS. 14-18 to aid in understanding the descriptions that follow. The x-, y-, and z-axes have been assigned as follows. The x-axis is perpendicular to the longitudinal axis of the vertebral column and perpendicular to the coronal/frontal plane (i.e., x-axis defines anterior vs. posterior relationships), The y-axis runs substantially parallel to the vertebral column and perpendicular to the transverse plane (i.e., y-axis defines superior vs. inferior relationships) The z-axis is perpendicular to the longitudinal axis of the vertebral column and perpendicular to the median/midsagittal plane (i e., z-axis defines the lateral right and left sides of body parts). The set of coordinate axes (x-, y-, and z-axes) are consistently maintained throughout although different views of vertebrae and the spinal column may be presented.

It is to be understood that the median/midsagittal plane passes from the top to the bottom of the body and separates the left and the right sides of the body, and the spine, into substantially equal halves (e.g., two substantially equal lateral sides). Further, it is to be understood that the frontal/coronal plane essentially separates the body into the forward (anterior) half and the back (posterior) half, and is perpendicular to the median plane. Still further, it is to be understood that the transverse plane is perpendicular to both the median plane and coronal plane and is the plane which divides the body into an upper and a lower half.

Referring briefly to FIG. 14, vertebra 110 includes a vertebral body 112 and a vertebral foramen 115 containing a portion of the ligamentum flavum 126, spinal cord 128, and an epidural space 127 between ligamentum flavum 126 and spinal cord 128. Spinal cord 128 comprises a plurality of nerves 134 surrounded by cerebrospinal fluid (CSF) contained within dural sac 132. Nerves 134 normally comprise only a small proportion of the dural sac 132 volume. Thus, CSF filled dural sac 132 is somewhat locally compressible, as localized pressure causes the CSF to flow to adjacent portions of the dural sac. Epidural space 127 is typically filled with blood vessels and fat. The posterior border of the normal epidural space 127 generally defined by the ligamentum flavum 126, which is shown in its normal, non-thickened state in FIG. 14.

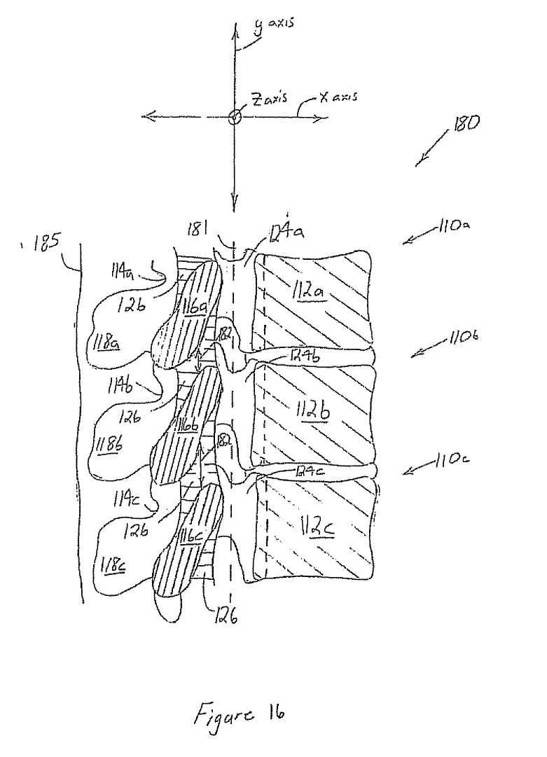

FIG. 15 illustrates a case of spinal stenosis resulting from a thickened ligamentum flavum 126. Since vertebral foramen 115 is defined and surrounded by the relatively rigid bone its volume is essentially constant. Thus, thickening of ligamentum flavum 126 within vertebral foramen 115 may eventually result in compression of spinal cord 128. In particular, the thickened ligamentum flavum 126 may exert a compressive force on tie posterior surface of dural sleeve 132. In addition, thickening of ligamentum flavum 126 may compress the blood vessels and fat occupying epidural space 127,

Compression of spinal cord 128, particularly in the lumbar region, may result in low back pain as well as pain or abnormal sensations in the legs. Further, compression of the blood vessels in the epidural space 127 that houses the nerves of the cauda equina may result in ischemic pain termed spinal claudication.

In order to relieve the symptoms associated with a thickened or enlarged ligamentum flavum 126, a variety of suitable procedures and techniques may be employed to reduce the size of the thickened/enlarged ligamentum flavum 126, thereby decompressing spinal cord 128 as well as blood vessels contained within the epidural space 127. Examples of suitable decompression techniques include without limitation, removal of tissue from ligamentum flavum 126, laminectomy, laminotomy, and retraction and anchoring of ligamentum flavum 126, U.S. Patent Application Ser. Nos. 60/747,166, 11/193,581, 60/733,754, 60/733,819, 60/733,685, 60/7.33,849, 60/733,552, 60/747,166, each of which is hereby incorporated herein by reference in its entirety, discloses several methods, techniques, tools, and devices that may be used to treat spinal stenosis caused by an enlarged ligamentum flavum by excising portions of the enlarged ligamentum flavum.

Accessing ligamentum flavum 126 with a tissue excision device to remove portions of ligamentum flavum 126 can present significant challenges. For instance, in some conventional approaches to correct stenosis caused by an enlarged ligamentum flavum, an incision is made in the back of the patient and then the muscles and supporting structures of the vertebral column (spine) are stripped away, exposing the posterior aspect of the vertebral column. Subsequently, the thickened ligamentum flavum is exposed by removal of a portion of vertebral arch 114, often at lamina 116, which encloses the anterior portion of the spinal canal (laminectomy). The thickened ligamentum flavum ligament 126 can then be excised by sharp dissection with a scalpel or punching instruments. However, this approach is usually performed under general anesthesia and typically requires an extended hospital stay, lengthy recovery time and significant rehabilitation. Referring briefly to FIG. 15, as another example, some MILD procedures access ligamentum flavum 126 percutaneously by boring a hole through the vertebral arch 114 of vertebra 110, often through a lamina 116. However, while such a MILD approach is minimally invasive and reduces recovery time, such an approach requires the additional step of boring a hole in the posterior of the vertebra 110 of interest. Thus, in some cases it will be preferable to employ a MILD that percutaneously accesses ligamentum flavum 126 without the need to cut or bore through the vertebrae However, embodiments of percutaneous tissue access device 100 described herein may be employed to sufficiently provide percutaneous access to a particular region of the enlarged ligamentum flavum 126 to be excised.

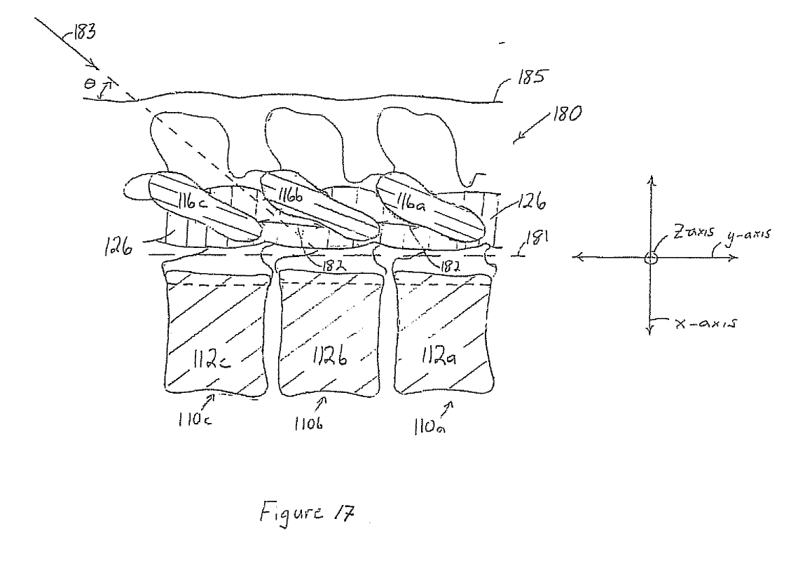

FIG. 16 is a partial cross-sectional lateral view of a segment of a vertebral column 180. The segment of vertebral column 180 illustrated in FIG. 16 includes three vertebrae 110a, 110b, and 110c. Each vertebra 110a, 110b, 110c includes a vertebral body 112a, 112b, 112c, that supports a vertebral arch 114a, 114b, 114c, respectively. Vertical body 112a, 112b) 112c is anterior to vertebral arch 114a, 114b, 114c, respectively. Each vertebral arch 114a, 114b, 114c together with vertebral body 112a, 112b, 112c, respectively, encloses a vertebral foramen 115a, 115b, 115c. The succession of vertebral foramen 115a, 115b, 115c in adjacent vertebrae 110a, 110b, 110c define vertebral canal 181 (spinal canal) that runs along the length of vertebral column 180. Vertebral canal 181 contains the spinal cord (not shown in FIG. 16).

Each vertebral arch 114a, 114b, 114c includes two pedicles 124a, 124b, 124c, which project posteriorly to meet two lamina 116a, 116b, 116c, respectively. It is to be understood that in this view, one pedicle has been removed from each vertebra 110a, 110b, 110c and only the cross-section of one lamina 116a, 116b, 116c is visible. The two lamina 116a, 116b, 116c meet posteriomedially to form the spinous process 115a, 115b, 115c, respectively

Lamina 116a, 116b, 116c of adjacent vertebra 110a, 110b, 110c are connected by ligamentum flavum 126 (shown in cross-section). The relatively elastic ligamentum flavum 126 extends almost vertically from superior lamina to inferior lamina of adjacent vertebrae. In particular, ligamentum flavum 126 originates on the inferior surface of the laminae of the superior vertebrae and connects to the superior surface of the laminae of the inferior vertebrae. For instance, ligamentum flavum 126 originates on the inferior surface of lamina 116a of superior vertebra 110a and connects to the superior surface of lamina 116b of the inferior vertebra 110b. Thus, ligamentum flavum 126 spans an interlaminar space 182 (i.e., space between laminae of adjacent vertebrae). Interlaminar space 182 is generally the space between laminae of adjacent vertebrae in spinal column 180.

Still referring to FIG. 16, each lamina 116a, 116b, 116c comprises a relatively broad flat plate of bone that extends posteromedially and slightly inferiorly from pedicles 124a, 124b, 124c, respectively. Along the length of vertebral column 180, the lamina 116a, 116b, 116c overlap like roofing shingles, with each lamina substantially parallel to and at least partially overlapping the adjacent inferior lamina. Further, the adjacent substantially parallel laminae are separated by the intervening ligamentum flavum 126 and interlaminar space 182. For instance, lamina 116a is substantially parallel to and partially overlaps adjacent inferior lamina 116b and is separated from lamina 116b by ligamentum flavum 126 and interlaminar space 182.

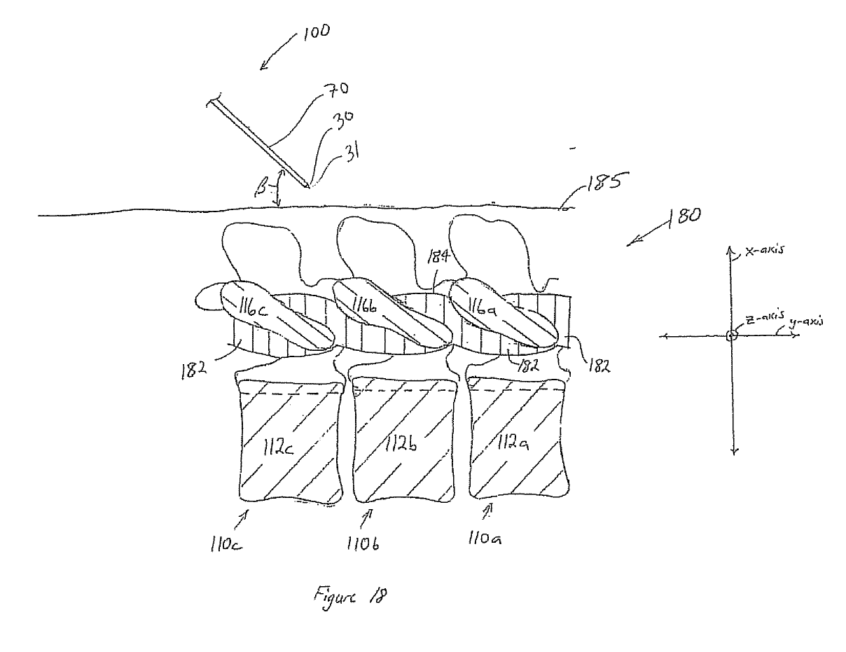

FIG. 17 illustrates vertebral column 180 as it may be oriented with the anterior side positioned down and posterior back surface 185 positioned upward, as may be encountered during a spinal procedure or surgery. In addition, in the embodiment illustrated in FIG. 15, ligamentum flavum 126 is thickened/enlarged, resulting in spinal stenosis. In particular, the anterior portions of enlarged ligamentum flavum 126 are extending into spinal canal 181, potentially exerting compressive forces on the spinal cord (not shown) that resides within spinal canal 181.

As previously discussed, to relieve compressive forces on the spinal cord and hence relieve the associated symptoms of spinal stenosis, portions of ligamentum flavum 126 may be excised. However, to percutaneously excise portions of ligamentum flavum 126 via minimally invasive techniques, the innate structure of vertebral column 180 and each vertebra may present significant imaging challenges. For instance, lateral imaging windows/views of ligamentum flavum 126 substantially in the direction of the z-axis may be obscured by the various processes of the vertebrae (e.g., transverse processes, superior articular processes, inferior articular processes), the laminae of each vertebra, etc. Further, some anterior-posterior (A-P) imaging windows/views of ligamentum flavum 126 substantially in the direction of the x-axis may also be obscured by the laminae. In particular, in the A-P radiographic imaging planes substantially in the direction of the x-axis, the posterior edges of parallel laminae overlap and obscure ligamentum flavum 126 and interlaminar space 182, particularly the anterior portions of ligamentum flavum 126 and interlaminar space 182 closest to spinal canal 181. However, with an imaging window/view in a plane substantially parallel to the X-Y plane, at an angle θ generally in the direction of arrow 183, and slightly lateral to the spinous process, interlaminar space 182 and ligamentum flavum 126 may be viewed without significant obstruction from neighboring laminae. In other words, imaging windows/views generally aligned with arrow 183 (FIG. 16) allow a more direct view of interlaminar space 182 and ligamentum flavum 126 from the posterior back surface with minimal obstruction by the vertebrae, laminae in particular.

Typically, the long axes of the substantially parallel laminae (e.g., laminae 116a, 116b, 116c) and interlaminar spaces (e.g., interlaminar spaces 182) are generally oriented between 60 and 75 degrees relative to posterior back surface 185. Thus, preferably the imaging means (e.g., x-ray beam, fluoroscopy tube, etc) is positioned generally in the direction represented by arrow 183, where θ is substantially between 60 and 75 degrees relative to the anterior back surface 185. In other words, the imaging means is positioned substantially parallel to the surface of the laminae. The resulting imaging window/view, termed “caudal-cranial posterior view” hereinafter, permits a clearer, more direct, less obstructed view of interlaminar space 182 and ligamentum flavum 126 from the general posterior back surface 185. The caudal-cranial posterior view permits a relatively clear view of interlaminar space 182 and ligamentum flavum 126 in directions generally along the y-axis and z-axis. However, the caudal-cranial posterior view by itself may not provide a clear imaging window/view of interlaminar space 182 and ligamentum flavum 126 in directions generally along the x-axis In other words, the caudal-cranial posterior view by itself may not provide a clear imaging window/view that can be used to accurately determine the posterior-anterior depth, measured generally along the x-axis, of a device across the ligamentum flavum 126,

Thus, in preferred embodiments, an additional imaging window/view, termed “caudal-cranial posterior-lateral view” hereinafter, is employed to provide a clearer, unobstructed view of interlaminar space 182 and ligamentum flavum 126 in directions generally along the y-axis and z-axis. The caudal-cranial posterior-lateral view is generated by orienting an imaging means generally at an angle θ relative to outer surface of the patient and also angling such imaging means laterally in an oblique orientation, revealing a partial lateral view of interlaminar space 182 occupied by ligamentum flavum 126 on the anterior side of the lamina and posterior to the underlying dural sac (not shown) and spinal cord (not shown).

By employing at least one of the caudal-cranial posterior view and the caudal-cranial posterior-lateral views, relatively clear imaging windows/views of the interlaminar space 182 and ligamentum flavum 126 in directions along the x-, y-, and z-axes may be achieved.

FIG. 18 illustrates vertebral column 180 and a partial view of an embodiment of percutaneous tissue access device 100 described herein with reference to FIGS. 1-13. Once unobstructed imaging windows/views of interlaminar space 182 and ligamentum flavum 126 are established in the manner described above, device 100 is employed to percutaneously access interlaminar space 182 and ligamentum flavum 126.

More specifically, using images of the interlaminar space 182 and ligamentum flavum 126 obtained from the desired direction(s), (e.g., caudal-cranial posterior view and the caudal-cranial posterior-lateral view), device 100 is employed to penetrate the skin and soft tissue in the posterior back surface 185 of the patient. In preferred embodiments, the skin entry point for tip 31 and device 100 is between 5 and 10 cm inferior (caudal to) the posterior surface of the interlaminar space 182 of interest. For instance, if the portion of ligamentum flavum 126 between lamina 116a and lamina 116b is the area of interest, then tip 31 and device 100 may be inserted into the patient's back about 5 to 10 cm inferior to posterior surface 84 of interlaminar space 182.

Referring still to FIG. 18, tip 31 of device 100 is preferably initially inserted into the posterior tissue and musculature of the patient generally parallel to the longitudinal axis of spinal column 180. In other words, the angle β between the posterior back surface 185 and device 100 is between 0 and 10 degrees when device 100 is initially inserted. Further, device 100 is preferably inserted into the posterior tissue and musculature of the patient on the same side (ipsilateral) of the median plane as the area of interest (e.g., the targeted portion of ligamentum flavum 126). Once device 100 is inserted into the posterior tissue and musculature of the patient, device 100 then may be oriented 5 to 90 degrees relative to the posterior back surface 185 in order to create a trajectory across ligamentum flavum 126 in the area of interest. It is to be understood that once device 100 is inserted into the patients posterior back surface 185, handle 20 and tip 31 of device 100 are moderately free to pivot about the insertion location in posterior back surface 185 in the general direction of the y-axis and the z-axis, and may be advanced posteriorly or anteriorly generally in the direction of the x-axis.

Once inserted into the posterior tissue and musculature of the patient, device 100 can be positioned to provide a trajectory across interlaminar space 182 in the area of interest, generally towards the anterior surface of the lamina superior to the area of interest. For example, if interlaminar space 182 between lamina 116a and lamina 116b is the area of interest, cannula 70 of device 100 is preferably positioned to provide a trajectory that will allow a cutting instrument to be inserted through bore 75 of cannula 70 across interlaminar space 182 between lamina 116a and lamina 116b towards the anterior surface of lamina 116a (superior lamina).

By switching between the caudal-cranial posterior view and the caudal-cranial posterior-lateral view, or by viewing both the caudal-cranial posterior view and the caudal-cranial posterior-lateral view at the same time, device 100 can be advanced to ligamentum flavum 126 in the area of interest with more certainty than has heretofore been present. Once device 100 has reached ligamentum flavum 126, the user may unlock cannula 70 from handle 20 as previously described and withdraw handle 20 and elongate member 30, leaving cannula 70 adequately positioned to provide access via bore 75 to the portions of ligamentum flavum 126 to be excised. Following removal of elongate member 30 from bore 75 of cannula 70, a variety of tissue excision devices may be advanced through bore 75 toward ligamentum flavum 126 and inserted in ligamentum flavum 126 in the region of interest to excise tissue therefrom. In some embodiments, excision can be performed generally from posterior to anterior across interlaminar space 182 and then laterally along the anterior portion of ligamentum flavum 126 if desired. The actual depth of the tip of any tissue excision device passing through bore 75 of cannula 70 in the general direction of the x-axis may be adjusted with guidance from the caudal-cranial posterior-lateral view and appropriate retraction/advancement of the excision device and appropriate adjustment of cannula 70 between 5 and 90 degrees relative to the posterior back surface 185.

In the manner described, device 100 may be used to provide access to a thickened ligamentum flavum. Once device 100 is employed to properly position cannula 70, a variety of tools may be inserted through bore 75 of cannula 70 to access and excise portions of the thickened ligamentum flavum, thereby reducing compression of the spinal cord.

While preferred embodiments of this invention have been shown and described, modifications thereof can be made by one skilled in the art without departing from the scope or teaching of this invention. For example, the means by which the safety zone is formed may be varied, the shape and configuration of the tissue excision devices may be varied, and the steps used in carrying out the technique may be modified. Accordingly, the invention is not limited to the embodiments described herein, but is only limited by the claims that follow, the scope of which shall include all equivalents of the subject matter of the claims. Likewise, the sequential recitation of steps in a claim, unless explicitly so stated, is not intended to require that the steps be performed in any particular order or that a particular step be completed before commencement of another step.

What is claimed is:

1. A surgical device for percutaneously accessing a tissue of interest comprising:
a handle including a longitudinal groove;
a resilient member disposed within the groove;
a locking sleeve at least partially disposed within the groove and slidingly engaging the groove, wherein the locking sleeve has a first end engaging and compressing the resilient member and a second end extending beyond the groove, and wherein the second end includes a flange defining a locking recess having a non-circular cross-section;
a cannula including a locking end and an axial through bore, wherein the locking end includes a locking flange having a non-circular cross-section;
wherein the non-circular cross-section of the locking flange and the non-circular cross-section of the locking recess are adapted to prevent the rotation of the cannula relative to the locking sleeve when the locking flange is at least partially received by the locking recess; and
means for rotationally coupling the cannula to the handle.

2. The surgical device of claim 1 further comprising an elongate member extending axially from the handle.

3. The surgical device of claim 2 wherein the elongate member comprises a trocar.

4. The surgical device of claim 1 wherein the handle includes an aperture through a lateral side of the handle to the groove, and wherein the locking sleeve includes a projection extending radially therefrom and adapted to slidingly engage the aperture.

5. The surgical device of claim 4 wherein the engagement of the projection with the aperture restricts the rotation of the locking sleeve relative to the handle.

6. The surgical device of claim 5 wherein engagement of the projection with the aperture restricts the locking sleeve from exiting the groove.

7. The surgical device of claim 1 wherein the resilient member comprises a spring disposed within the groove.

8. The surgical device of claim 1 wherein the non-circular cross-section of the locking flange and the non-circular cross-section of the locking recess comprise mating octagonal cross-sections.

9. The surgical device of claim 1 wherein the surgical device has a locked position with the locking flange at least partially received by the locking recess, and the locking end of the cannula rotationally coupled to the handle.

10. The surgical device of claim 9 wherein the surgical device has an unlocked position with the cannula rotationally decoupled from the handle.

11. The surgical device of claim 2 wherein the surgical device has a locked position with the elongate member coaxially disposed within the axial through bore of the cannula, the locking flange at least partially received by the locking recess, and the cannula rotationally coupled to the handle.

12. The surgical device of claim 11 wherein the surgical device has an unlocked position with the elongate member withdrawn from the axial through bore of the cannula.

13. A surgical device for percutaneously accessing a tissue of interest comprising:
a handle including an axial annular groove defining an axial post, wherein the axial post has a threaded portion;
a resilient member disposed within the annular groove;
an annular locking sleeve partially disposed within the annular groove, wherein the annular locking sleeve has a first end engaging and compressing the resilient member and a second end extending beyond the annular groove, wherein the second end includes a flange having a locking recess with a noncircular cross-section; and
a cannula including a locking end and an axial through bore, wherein the locking end includes a locking flange having a non-circular cross-section, and wherein the axial through bore has an threaded portion adapted to mate with the threaded portion of the axial post;
wherein the non-circular cross-section of the locking flange and the non-circular cross-section of the locking recess are adapted to prevent the rotation of the cannula relative to the locking sleeve when the locking flange is at least partially received by the locking recess;
wherein the surgical device has a locked position with the cannula threadibly coupled to the axial post and the locking flange at least partially received within the locking recess.

14. The surgical device of claim 13 further comprising an elongate member extending axially from the axial post.

15. The surgical device of claim 14 wherein the elongate member is coaxially disposed within the axial through bore of the cannula when the surgical device is in the locked position.

16. The surgical device of claim 15 wherein the surgical device has an unlocked position with the elongate member withdrawn from the axial through bore of the cannula.

17. The surgical device of claim 14 wherein the elongate member comprises a trocar.

18. The surgical device of claim 13 wherein the handle comprises a radial aperture through a lateral side of the handle to the annular groove, and wherein the annular locking sleeve includes a projection extending radially therefrom adapted to slidingly engage the aperture in the handle.

19. The surgical device of claim 17 wherein the engagement of the projection and the aperture restricts the rotation of the annular locking sleeve relative to the axial post.

20. The surgical device of claim 18 wherein engagement of the projection and the aperture restricts the annular locking sleeve from completely exiting the annular groove.

21. The surgical device of claim 9 wherein the surgical device has an unlocked position with the threaded portion of the post decoupled from the threaded portion of the axial through bore of the cannula.

22. A surgical device for percutaneously accessing a tissue of interest comprising:
a handle including an axial annular groove defining an axial post;
a resilient member disposed within the annular groove;
an annular locking sleeve at least partially disposed within the annular groove and slidingly engaging the axial post, wherein the annular locking sleeve has a first end engaging and compressing the resilient member and a second end extending beyond the annular groove;
a cannula including a locking end and a central axial bore;
means of rotationally coupling the locking end of the cannula to the post; and
means of restricting the rotation of the locking end relative to the annular sleeve.
